(19) United States
(12) Patent Application Publication
(10) Pub. No.: US 2014/0100651 A1
(43) Pub. Date: Apr. 10, 2014

(54) Medical Device Fastener Mechanisms

(71) Applicant: California Institute of Technology, (US)

(72) Inventors: Arash Kheradvar, Irvine, CA (US); Jimmy L. Su, Irvine, CA (US); Ahmad Falahatpisheh, Irvine, CA (US); Morteza Gharib, Altadena, CA (US)

(73) Assignee: California Institute of Technology, Pasadena, CA (US)

(21) Appl. No.: 13/773,389

(22) Filed: Feb. 21, 2013

Related U.S. Application Data
(60) Provisional application No. 61/601,470, filed on Feb. 21, 2012.
(60) Provisional application No. 61/650,813, filed on May 23, 2012.

(51) Int. Cl. A61F 2/24

(52) U.S. Cl. CPC A61F 2/2409; A61F 2/24
USPC 623/2.1

(57) ABSTRACT

Connecting mechanisms between medical device or other physical components are described. One or more plug pin connector(s) is received in a matching socket connector(s) to either permanently or temporarily join at least two components together along with associated components. Each plug connector is associated with a releasable pull line to facilitate such assembly.

### RELATED APPLICATIONS

This filing claims the benefit of and priority to U.S. Patent Application No. 61/601,470 filed Feb. 21, 2012 and U.S. Patent Application Ser. No. 61/650,813 filed May 23, 2012, each of which applications are incorporated by reference herein in its entirety.

### FIELD

The embodiments described herein optionally relate to medical device assembly features, such as for percutaneously-delivered heart valves or otherwise.

### BACKGROUND

Medical devices often require some measure of assembly. Certainly, this is the case when the construction includes each of a support structure and a biological graft or synthetic tissue construction such as in the case of many prosthetic valves. A need exists for conveniently, accurately and securely applied fasteners that may substitute for the use of sutures in prosthetic heart valve applications and others.

### SUMMARY

The invention embodiments address the stated need and others, especially in medical device applications, where the various features described herein may prove advantageous. Inventive aspects hereof include the embodiments described, methods/processes of their use, products by such processes and larger assemblies incorporating such embodiments.

Embodiments comprise a connecting mechanism/device that joins together at least two components via a pulling line advantageously used to join and secure the components together. The system also allows the pulling mechanism to be released.

The fastener mechanism comprises at least one plug pin connector (alternatively referred to as a fastener itself), that is brought together by a pulling system into a matching connecting socket to, either permanently or temporarily, join at least two components together. The pulling mechanism, such as, but not limited to at least one line, wire, cable, thread, string, etc, composed of a material such as, but not limited, to Nitinol, nylon, silk, polypropylene or some other string-like material, is connected to the plug-pin-like connector and directed towards the matching socket connector. The socket may be formed in the frame of a stent-like support for a valve and used to secure a graft section thereto. Other configurations are possible as well.

The pulling mechanism is operated either spontaneously or manually at a user-defined rate to bring the aforementioned components together. As the plug pin connector is linked to the matching socket connector section, the plug-like component locks itself into place so as to prevent removal or separation of the two aforementioned components. Simultaneously, or immediately after the instant of locking (in either case, upon or after linking), the pulling mechanism is spontaneously or manually released from the pin, allowing the pull line to be separated and/or recovered from the conjoined component assembly.

The plug pin connector/fastener may be shaped as a conical plug which is inserted into a matching hole. The plug connector can be formed of, but not limited to, a deformable yet elastic material such as, but not limited to, Nitinol (whether set for superelastic Nitinol, or shape memory use) plastic biocompatible polymers (e.g., PTFE, FEP, PEEK, PI, etc.) or be made of other material. It may formed in such a way, such as, but not limited to, a conical shape to guide connection of the plug fastener towards the matching connector socket. Optionally, the mechanism is flexible enough to allow the pin connector to deform when inserted into the matching hole (i.e., socket connector), and then recover into an appropriate shape once the plug pin connector has been, depending on the application, fully or partially inserted through the socket connector hole in order so that the fastener cannot be removed or separated.

Deformation of the plug pin connector through the socket hole optionally allows some degree of shape configuration change that triggers the release, discharge or cut of the pulling mechanism. In one variation, the tip of the plug connector is formed with at least one or more fin-like structures that close together to form a different shape, such as, but not limited to, a conical configuration, yet allows a pull-wire to pass between the fins. Also, there may be a compartment between the fins that can contain an object, larger than the cross-section of the wire-like mechanism being used, such as, but not limited to, a spherical ball. This object, or relatively enlarged termination of the pull line is firmly connected to the end of the same, and being larger than the cross-sectional area of the pull line/wire, is trapped within the pin fastener compartment, as long as the fin-like structures at the tip of the pin fastener are kept at a distance smaller than the diameter of the object.

In use, the fins are kept closed together prior to insertion within the matching socket connector hole, at which point, deformation of the plug connector may allow allows sufficient space for the object to escape, subsequently allowing the release, discharge or cut of the pull-wire mechanism. Alternatively, the release of the pull line may be triggered by the partial or full locking of the plug connector and the matching socket. As an example, the aforementioned fin-like structures may be separated by the trigger of a spring-like mechanism, which allows the release, discharge, or cut of the pull line mechanism.

In another embodiment, at a determined time after locking has occurred, the pulling mechanism is manually released from the pin, allowing the pulling mechanism to be separated and/or recovered from the conjoined component assembly. In which case, the pulling mechanism may be connected to the pin fastener through a physical wire loop that allows the pulling mechanism to be inserted through the pin fastener and doubled back out through the pin. Release, discharge or removal of the pulling mechanism in such an embodiment is accomplished through the cutting of the line/wire which allows it to be separated from the plug fastener and overall construction.

Portions of the plug connector may formed such as, but not limited to, an annular snap ring with a conical end configuration and include a center hole that allows a pull-wire/line to pass between the conical snap. As such, the conical snap may include a channel or pathway cut or otherwise formed along its length in order to allow the pulling mechanism to travel in scenarios where the pulling force occurs at an angle of, such as, but not limited to, 90 degrees relative to the pin insertion direction.

A medical device (e.g., a prosthetic valve) assembly is advantageously produced employing any of the subject fastener embodiments. In the case of a valve-type device, the plug connector may be pulled into connector sockets formed in a support frame for the valve, passing through and “sandwiching” valve leaflet material. Alternatively, the valve material may be secured to the to the plug connector body (e.g. by suture, medical adhesive or other means) and then the sub-assembly attached to the support frame. Irrespective of the means for any other material attachment, the pull line used for attaching the plug and socket connectors may include a separate or integral needle tip (as typical with sutures) for threading through the tissue-emulating portion of the construction.

### BRIEF DESCRIPTION OF THE DRAWINGS

The figures diagrammatically illustrate inventive embodiments. Variations other than those shown in the figures are contemplated as described in a broader sense per the Summary above, as generically claimed, or otherwise.

FIGS. 1A-1C are perspective views of a finned plug pin connector being inserted and locked into a matched socket connecting hole without the referenced pull-wire/line mechanism.

FIG. 2 is a perspective view of a prosthetic valve with its frame and leaflet construction as may be secured together with the subject fastener arrangements, with one option for the same provided in a detail top view.

FIGS. 3A-3D are side cross-section views illustrating an example of a pull line retention and release mechanism embodiment.

FIGS. 4A and 4B are side cross-section views illustrating a spring loaded example of a pull line retention and release mechanism.

FIG. 5 is a side view of another plug connector embodiment, again, without the pull line shown.

FIGS. 6A-6C are perspective views of the plug connector arrangement of FIG. 5 being inserted and locked into two matched socket connecting holes.

FIGS. 7A-7C are side views and FIGS. 8A-8C are perspective views of the pull line release mechanism for the construct shown in FIG. 6C in stages of use/release.

FIGS. 9A and 9B are side and top detail views of the FIG. 5 plug connector/fastener embodiment.

### DETAILED DESCRIPTION

Various exemplary embodiments are described below. Reference is made to these examples in a non-limiting sense, as it should be noted that they are provided to illustrate more broadly applicable aspects of the devices, systems and methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present invention. All such modifications are intended to be within the scope of the claims made herein.

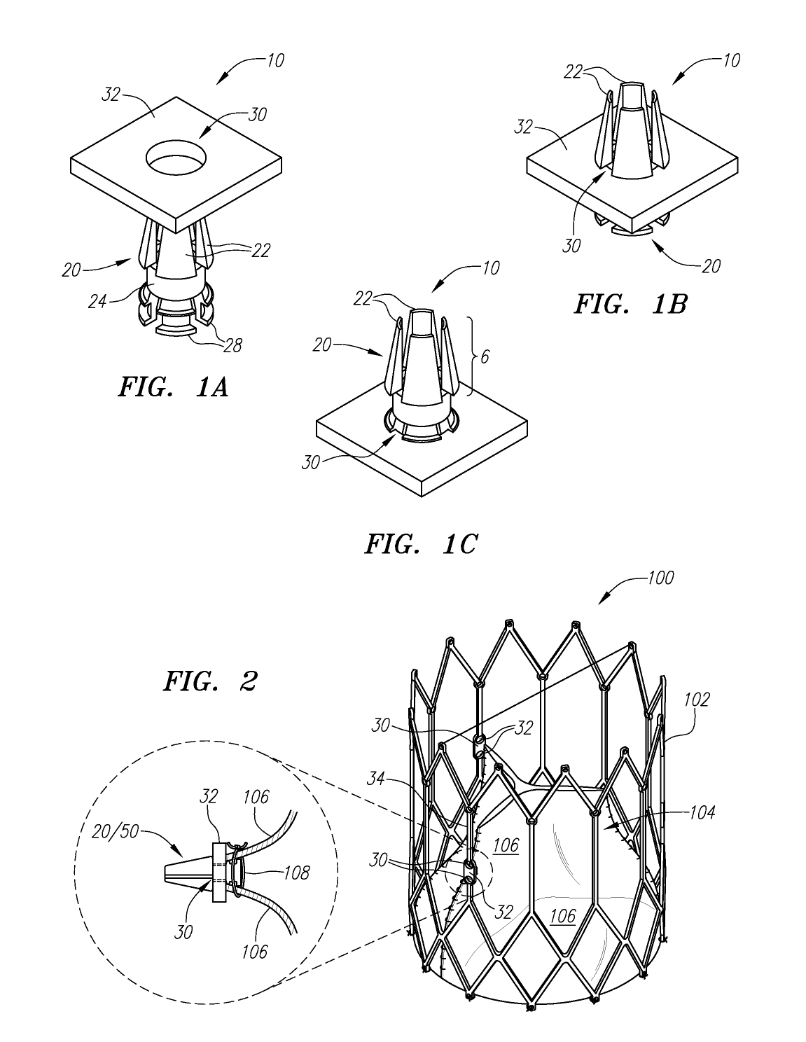

FIGS. 1A-1C are perspective views of a fastener system 10 according to one embodiment. In each figure, a plug connector 20 and a socket connector 30 defined as a hole in a section of material 32 is shown. Notably, material 30 may a single plane of material or a multi-layered construct to be held together by engagement/linking of the connector portions.

Plug connector is illustrated with a plurality of fins 22 arranged to define a conical taper “C” as a lead-in to socket connector 30. An annular suction 24 visible in FIG. 1A locks in place as shown in FIG. 10, stopping at flange 26. Given flexibility imparted by the annulus-side struts/fins 28 it may be possible to compress and release the connectors once linked.

In any case, FIG. 2 illustrates an artificial valve 100 for percutaneous delivery in a partially assembled state. It includes a stent frame 102 and valve 104 comprising leaflets 106. The referenced stent frame may be constructed from Nitinol hypotube using conventional laser cutting and electropolishing techniques and/or be otherwise constructed. Various delivery system components and other options for such devices may be appreciated in reference to PCT/US12/49645 filed Aug. 3, 2012, incorporated herein by reference in its entirety.

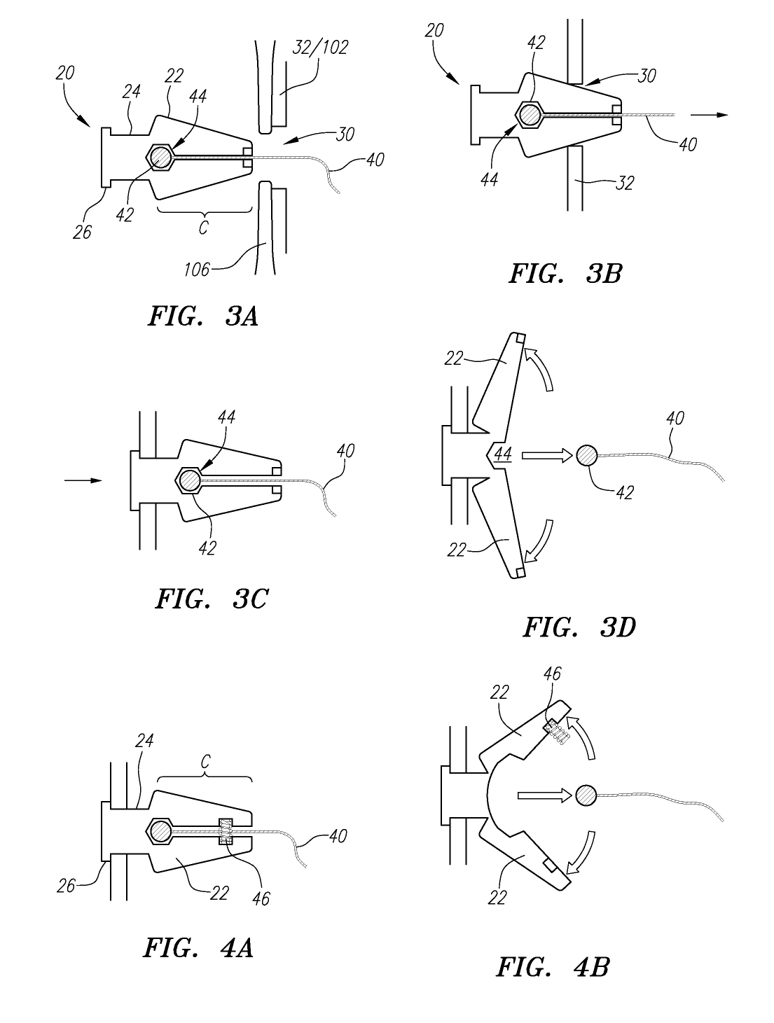

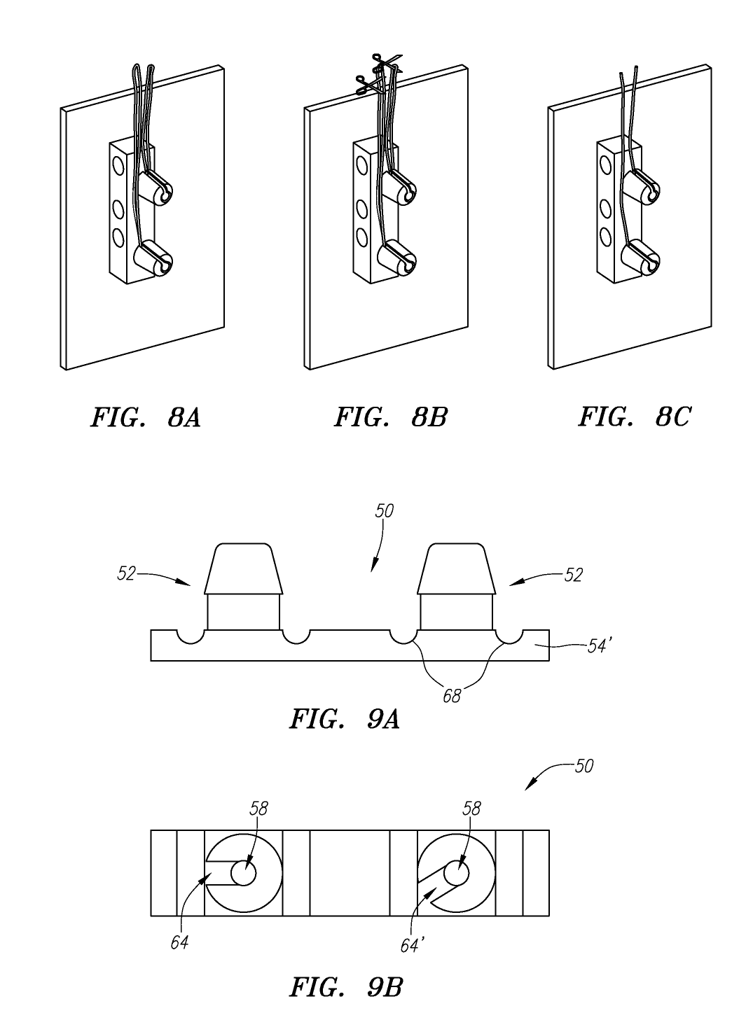

The detail view in FIG. 2 shows socket connector 30 and the material 32 it is formed with a plug connector 20/50 attached thereto. In this case, the socket is viewed along the axis of a stent frame strut 34. In this view, the valve material 106 is connected by suture 108 to the plug connector. Further description of options for such attachment is addressed in connection with FIGS. 5 and 9A. FIG. 3A also shows another manner of attaching graft material 106 to the frame material 32/102. Namely, it can be pierced and sandwiched using the flange/base 26 of the plug connector.

In any case, FIG. 3A illustrates the manner in which a plug connector 20 is pulled into socket 30 in frame 102 (or more generically material 32) using a pull line 40. As shown in FIGS. 3B and 3C, pull line 40 is able to draw the connector into a linked engagement with socket 30 given the attached object/end 42 of the pull line constrained in a pocket/cavity/chamber 44 within the plug connector. As shown in FIG. 3C, after the connectors are linked, fins 22 are able to open and release object 42 (be it a spherical attachment, an enlargement of the line produced with a laser-created blob, or body otherwise constructed) as chamber 44 is opened.

As illustrated in FIGS. 4A and 4B the operative action in FIG. 3D may be urged by an included spring 46 in a spring-loaded mechanism. A locking mechanism of any type known to those of skill in the art (such as a bail, clasp or other feature) may be incorporated in plug connector 20 so as to secure the spring-loaded mechanism until automatically unlatched upon linking with socket 30. As yet another alternative, a delivery sheath, specialized forceps/pliers, etc. may be used to constrain either the spring-loaded or a non spring-loaded plug connector during installation. Such techniques for device and subcomponent delivery and assembly are well known in the art.

FIG. 5 is a side view of another plug connector embodiment 50. Here, two plug connectors 52 are joined by a bridge or base 54. Ports or holes 56 may be provided therein for looping suture through for graft connection as described in FIG. 2. Also, through-holes or channels 58 are advantageously provided for pull lines (as later illustrated). Channels 58 may be angled within the base (e.g. comparing their axis of orientation to that of the plug connectors) to provide leverage when pulling plug connector 50 into linked arrangement (e.g., by upward/relatively aligned pulling of draw lines as illustrated in FIG. 7A) with socket material 32.

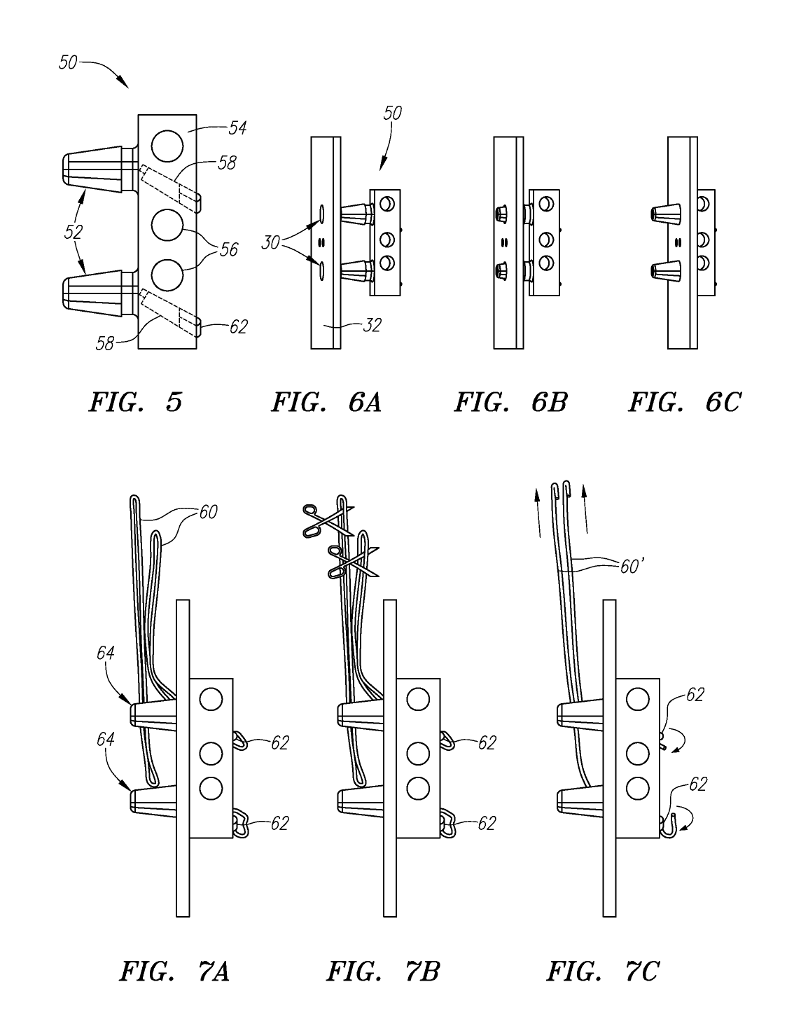

FIGS. 6A-6C illustrates such action. Namely: linking plug connector 50 with a matching-pattern set of socket connectors 30. This action is accomplished as illustrated in FIGS. 7A-7C and 8A-8C. Here, pull line loops 60 run around bars or bosses 62 incorporated in connector plug base 54. The pull line loops 60 pass through holes 48 and then through slots 64 in the plug connector 52. They are thus secured for use in pulling the plug connectors into linked engagement with the socket connector sections as shown in FIGS. 7A and 8A. Once accomplished, the loops may be cut as portrayed in FIGS. 7B and 8B. Then, the loops (now single strands 60′) may then be removed by pulling on one end as shown in FIGS. 7Ca and 8C.

Optional details of plug connector(s) suitable for such are shown in FIGS. 9A and 9B. In FIG. 9A, semi-circular troughs 68 are seen in side view. These may be used for centering/locating graft attachment suture 108. Without the inclusion of circular ports 56 (as in the previous embodiment), the height of the plug connection base 54′ can be reduced with no loss of functionality.

In FIG. 9B, a difference in the angular orientation of slots 64 and 64′ is seen in top view. The angling helps provide clearance for the lower loop 60 when pulling vertically as illustrated in FIGS. 8A-8C. More or less variation may be desirable for any given application.

Use of the fasteners as described and as may be further understood by those with skill in the art also form inventive embodiments hereof. The subject methods may include each of technician or physician assembly activities associated with implant preparation and/or its delivery, positioning, re-positioning, retrieval and/or release. Regarding these methods, including methods of manufacture and use, these may be carried out in any order of the events which is logically possible, as well as any recited order of events.

Furthermore, where a range of values is provided, it is understood that every intervening value, between the upper and lower limit of that range and any other stated or intervening value in the stated range is encompassed within the invention. Also, it is contemplated that any optional feature of the inventive variations described may be set forth and claimed independently, or in combination with any one or more of the features described herein.

Though invention embodiments have been described in reference to several examples, optionally incorporating various features, the invention is not to be limited to that which is described or indicated as contemplated with respect to each variation. Changes may be made to the variations described and equivalents (whether recited herein or not included for the sake of some brevity) may be substituted without departing from the true spirit and scope of the invention.

Reference to a singular item includes the possibility that there are a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the different inventive embodiments or aspects described herein is not to be limited to the examples provided and/or the subject specification, but rather only by the scope of the issued claim language. It should be understood, that the description of specific example embodiments is not intended to limit the invention to the particular forms disclosed, but on the contrary, this patent is to cover all modifications and equivalents as illustrated, in part, by the appended claims.

1. A fastener mechanism comprising:
a plug connector and a matching socket connector formed in a medical device, wherein the plug connector is adapted to link with a matching socket connector; and
a pull line attached to the plug connector.

2. The mechanism of claim 1, adapted for permanent one-way linking between the plug and socket connectors.

3. The mechanism of claim 1, adapted for releasable linking between the plug and socket connectors.

4. The mechanism of claim 1, wherein the plug connector is tapered toward an end and the pull line extends from the end.

5. The mechanism of claim 4, wherein the plug connector includes a conical shape portion and an annular snap lock portion.

6. The mechanism of claim 5, comprising a plurality of fins, wherein the pull line passes through a center of the conical shape.

7. The mechanism of claim 1, wherein the pull line is releasable from the plug connector.

8. The mechanism of claim 7, adapted to release the pull line upon linking the plug and socket connectors.

9. The mechanism of claim 8, wherein the pull line is secured to an object and the object is adapted to prevent the pull line from separating from the plug connector prior to linking the plug and socket connectors.

10. The mechanism of claim 9, wherein deformation of the plug connector allows release of the object.

11. The mechanism of claim 8, wherein the deformation also provides the linking.

12. The mechanism of claim 7, wherein the release is spring-actuated.

13. The mechanism of claim 7, wherein the pull line is releasable by cutting.

14. The mechanism of claim 13, comprising a plurality of plug connectors connected to a fist body in a pattern, and a plurality of socket connectors provided in a second body with the same pattern for linking the connectors to each other.

15. The mechanism of claim 14, wherein each plug connector incorporates a side channel for a pull line.

16. The mechanism of claim 15, wherein the pull line is looped through the plug connector.

17. The mechanism of claim 14, wherein each pull line passes through a channel in the plug connector body, and wherein an axis of each channel is angled with respect to an axis of each plug connector.

18. The mechanism of claim 1, wherein the plug and socket connectors are linked together to secure a valve segment of an artificial heart valve.

19. The mechanism of claim 18, wherein the pull line comprises a material selected from Nitinol, nylon, silk, and polypropylene.

20. A percutaneous artificial heart valve comprising:
a frame, a valve, at least one plug connector, and at least one matching socket connector,
wherein at least one segment of the valve is secured to the frame by a linked plug connector and matching socket connector.
